(19) United States
(12) Patent Application Publication
(10) Pub. No.: US 2005/0145243 A1
(43) Pub. Date: Jul. 7, 2005

(54) Nebuliser

(76) Inventor: Andrea Trombi, Parma (IT)

Correspondence Address: ARENT FOX PLLC, 1050 CONNECTICUT AVENUE, N.W., SUITE 400, WASHINGTON, DC, 20036, US

(21) Appl. No.: 10/511,265

(22) PCT Filed: Oct. 25, 2002
(86) PCT No.: PCT/IT02/00680

(30) Foreign Application Priority Data
May 28, 2002 (IT) PR2002A000027

(51) Int. Cl. A61M 11/00; B05B 17/06

(52) U.S. Cl. 128/200.14; 128/200.16; 128/200.21

(57) ABSTRACT

Nebuliser ampoule (2), in particular for aerosol therapy, of the type comprising an outlet (3) for dispensing a nebulised medical product, an element (4) for distributing the medical product and an element (5) for activating the nebulisation. The ampoule (2) is characterised in that the activator element (5) is physically separate from the element (4) for distributing the medical product. The distributor element (4) comprises a nozzle (6) for injecting a flow of air, called primary flow, inside the ampoule (2), said flow being necessary for generating the nebulisation, and at least a substantially conical body (7) inserted on the nozzle (6) and provided with at least a channel for conveying the medical product from a tank (8) of the ampoule (2) to a nebulisation area.

### TECHNICAL FIELD AND BACKGROUND ART

The present invention relates to a nebuliser ampoule, in particular for aerosol therapy, of the type comprising a mouthpiece for dispensing a nebulised medical product, an element for distributing the medical product and an element for activating the nebulisation.

As is well known, apparatuses for nebulising are used in particular in the field of aerosol therapy, i.e. of the therapeutic treatment of symptoms of the respiratory track, such as asthmatic or bronchial symptoms. Said therapeutic system provides for the generation of an aerosol, i.e. of a dispersion or nebulisation of appropriate medical liquids that act through the inhalation of the medical liquid itself.

Such apparatuses are widely used, especially in the case of paediatric therapies, and are provided in different formats able to meet different users' requirements. More specifically, nebulising apparatuses can also be constructed in portable formats, so that the user can have the necessary medicine available at any time, especially in the case of ailments entailing frequent or unpredictable respiratory crises, such as asthmatic ailments.

Pneumatic nebulising apparatuses also exist, so defined because they comprise a compressor that aspirates air from the environment and sends it to a nebulising ampoule containing the medical liquid.

Some pneumatic nebulisation apparatuses comprise, in addition to a primary conduit for the delivery of a flow of air necessary for nebulisation, and called primary flow, also a so-called supplementary, or secondary, channel, provided with an inlet through which ambient air enters by Venturi effect and because of the aspiration provided by the user during inspiration.

The flow of air of the secondary channel, called secondary flow, allows a better nebulisation of the medical product, in terms of quantity and quality of the generated spray.

During the expiration phase, the air exhaled by the user is expelled from the apparatus by means of an outlet.

Normally, both the inlet of the secondary channel and the outlet are provided with a valve, able to move between an opened position and a closed position to guarantee that the flow of air inside the apparatus is correctly directed, both during inspiration and during expiration. In particular, said valves are usually made of highly deformable plastic material and are actuated directly by the flow of air that impacts thereon.

Generally, the tank of the nebuliser ampoule is open superiorly and is provided inferiorly with a nozzle for the entry of the air coming from the primary delivery conduit of the compressor. On the nozzle is inserted a cone which deviates the flow of the air and draws the liquid from the tank through some channels, usually obtained on the cone itself. The cone-channels assembly is usually called medical product distributor element.

The channels are usually a pair of grooves obtained on opposite parts of the lateral surface of the cone, to enable the liquid to pass between the body of the cone and the nozzle for the entrance of the air inside the ampoule.

To enable the intake of the liquid through the channels, and consequently the nebulisation, a so-called nebulisation activator element is necessary.

In accordance with a possible prior art technique, said element is made in a single piece with the cone and is positioned in correspondence with an outlet of the nozzle.

The activator element is integral with a platelet with rectangular section, positioned parallel to the flow of air exiting the nozzle. Said platelet is connected to cone, generally by means of a pair of supports positioned on an edge of the cone itself in diametrical position. The flow of air coming from the primary conduit, impacting against the activator element, forms a pair of vortices that are able to generate such a turbulence as to create a vacuum in correspondence with the outlet of the nozzle.

In this way, the medical liquid is aspirated and rises along the channels present on the cone, mixing itself with the air of the delivery conduit (and possible with the air coming from the supplementary channel) and thereby forming the aerosol.

In regard to the compressor, it is generally housed in a rigid case, made for instance of plastic material, which incorporates the outlets of the intake and delivery conduits coming from the compressor itself. In use, mainly with traditional apparatuses, in particular for home use, the rigid case containing the compressor is usually set down on a plane whilst the nebulising ampoule is located in proximity to the user's face and is connected to the inlet of the delivery conduit by means of a flexible pipeline.

The compressor can comprise a header incorporating both the aspiration conduit and the delivery conduit, interfacing directly with the exterior by means of intakes obtained directly on the header itself and destined to be adapted to the profile of the rigid containment case.

Nebuliser ampoules provided with an activator element made of a single piece with the distributor have some important drawbacks.

First of all, because of the presence of the supports of the platelet, it is impossible to assure a flow of air that is substantially symmetrical relative to the axis of the primary conduit. Consequently, the nebulisation that is formed inside the ampoule is not homogeneous.

In the second place, the presence of the supports forces to construct a single pair of channels for aspirating the medical product. Given the geometry of the distributor and of the activator element, the supports inevitably interfere with at least a pair of channels positioned in correspondence with a diameter of the cone, compromising a correct distribution of the medical liquid inside the flow of air present in the ampoule.

### DISCLOSURE OF THE INVENTION

The aim of the present invention is to eliminate the aforesaid drawbacks making available a nebuliser ampoule provided with an activator element able to assure a primary flow of air, substantially symmetrical relative to the axis of the primary conduit.

Another aim of the present invention is to propose a nebuliser ampoule provided with a distributor element which allows to obtain any number of channels, regardless of the presence of the activator element.

An additional aim of the present invention is to make available a nebuliser ampoule provided with an activator element which does not interfere with the fluid dynamics of the spray in correspondence with the so-called aerosol generation plane, this term defining the space of the ampoule just outside the cone and around it.

Yet another aim of the present invention is to obtain a nebuliser ampoule provided with means for selecting the dimensions of the particles present in the spray, to improve the therapeutic effect of the medical product dispensed by the ampoule.

Said aims are fully achieved by the nebuliser ampoule, in particular for aerosol therapy, of the present invention, which is characterised by the content of the claims set out below and in particular in that the element for activating the nebulisation is physically separate from the element for distributing the medical product. The term “physically separate” means that the activator element is not made in a single piece with the distributor element and hence is distinct therefrom. However, it would be possible to interconnect the activator element and the distributor element, for instance by means of a snap-on coupling.

In particular, the distributor element comprises at least a nozzle for injecting a primary flow of air inside the ampoule to generate the nebulisation. The distributor element is provided with at least a preferably conical coating body, inserted on the nozzle and provided with at least a channel to convey the medical product from a tank of the ampoule to a nebulisation area.

### BEST MODE FOR CARRYING OUT THE INVENTION

These aims and other aims will become more readily apparent from the description that follows of a preferred embodiment illustrated, purely by way of non limiting example, in the accompanying drawing tables, in which:

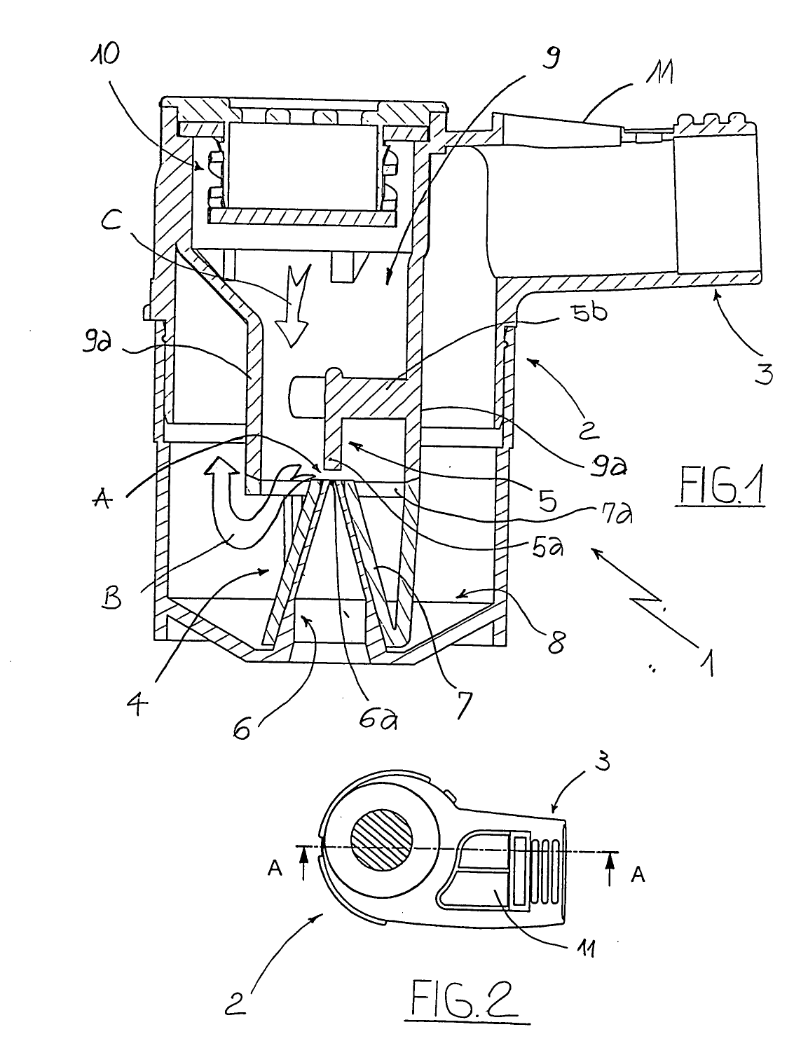

FIG. 1 shows a partially section front view of an apparatus for aerosol therapy provided with a nebuliser ampoule according to the invention;

FIG. 2 shows a top view of the apparatus of FIG. 1;

FIG. 3 shows a lateral view of the ampoule of the apparatus shown in FIG. 1;

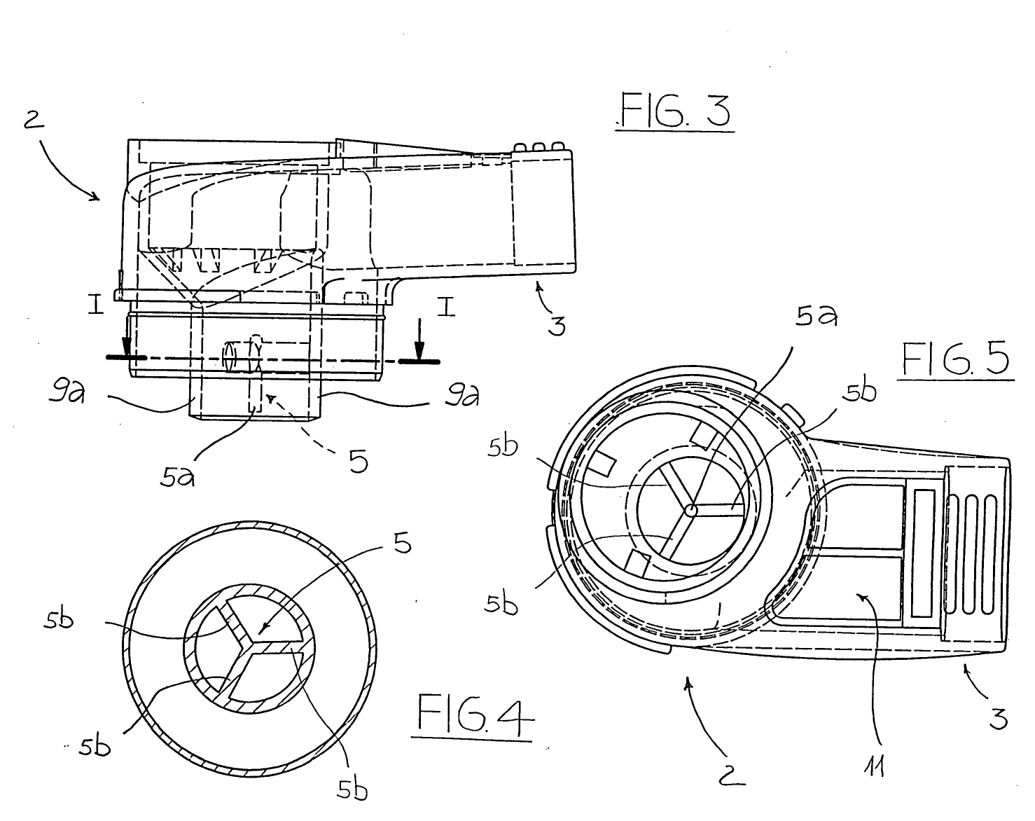

FIG. 4 shows a plan view of the ampoule of FIG. 3, enlarged and sectioned according to the trace I-I;

FIG. 5 shows a top view of the ampoule shown in FIG. 3, with some parts remove the better to highlight others;

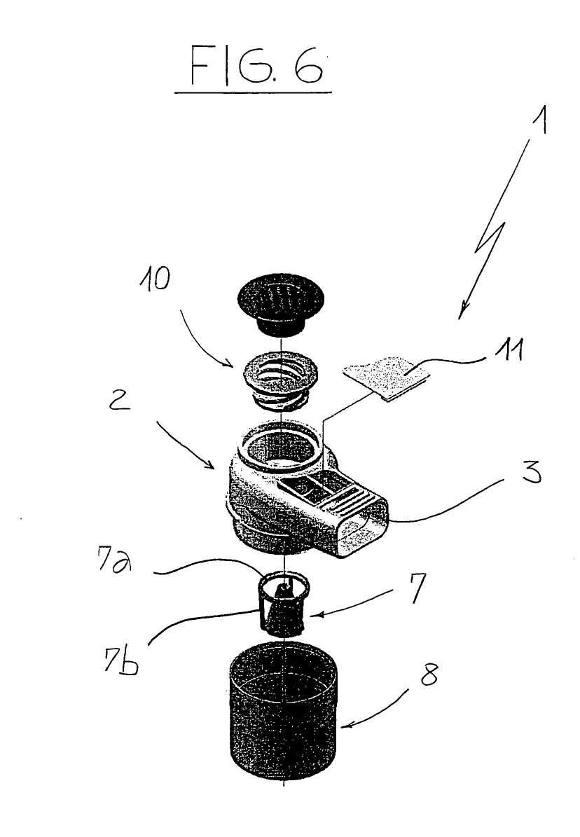

FIG. 6 shows an exploded view of the apparatus of FIG. 1.

With reference to the figures, the apparatus for aerosol therapy is globally indicated with the number 1 and comprises a nebuliser ampoule 2 provided with a mouthpiece 3 for dispensing a nebulised medical product.

The nebuliser ampoule 2 originally comprises an element 4 for distributing the medical product, physically separate from an element 5 for activating the nebulisation. The term “physically separate” means that the activator element 5 is not made in a single piece with the distributor element 4 and hence is distinct therefrom, but is able in any case to be interconnected thereto, for instance by means of snap-on coupling.

In the illustrated embodiment, the distributor element 4 comprises a nozzle 6 for injecting a primary flow of air inside the ampoule 2, said flow being necessary for generating the nebulisation.

The distributor element 4 comprises a coating body 7, preferably conical, inserted on the nozzle 6 and provided with at least a channel (not shown herein) for conveying the medical product from a tank 8 of the ampoule 2 to a nebulisation area. In particular, one or two channels can be obtained by means of a plurality of grooves preferably provided on opposite parts of a lateral surface of the coating body 7.

With particular reference to FIG. 6, the coating body 7 has portions 7a defining extensions of the lateral walls 9a of the secondary channel 9. In the illustrated embodiment, said portions consist of a ring connected to the coating body 7 by means of supporting element 7b. In particular, the ring is positioned in correspondence with the lower ends of the lateral walls 9a of the secondary channel.

In regard to the activator element 5, it has a portion 5a having substantially circular section. In particular, the activator element 5 is superposed to the nozzle 6 at a pre-set distance from an outlet 6a thereof.

The nebuliser ampoule 2 comprises a supplementary, or secondary, channel 9, for introducing a secondary flow of air inside the ampoule 2, in order to increase the nebulisation of the medical product. In the illustrated embodiment, the secondary channel 9 is coaxial to the distributor element 4 and is provided with lateral walls 9a that extend below an outlet 6a of the distributor element 4 or in any case below a plane for generating the nebulisation (indicated with the arrow A in FIG. 1). Specifically, the lateral walls 9a serve as means for selecting the particles that compose the nebulised medical product. In particular, the lateral walls 9a, together with the secondary flow of air, force the particles having larger dimensions to re-settle in the tank 8 of the ampoule 2. An aerosol formed by particles of small dimensions has better therapeutic effectiveness, because it penetrates in depth in the user's respiratory tract.

The activator element 5 is retained in position by means of a plurality of supports 5b connected to the secondary channel 9 and preferably made in a single body with the activator element itself. Specifically, said supports are arranged radially to the secondary channel and are preferably three. In the illustrated embodiment, the activator element 5 and the related supports 5b are made in a single piece with the secondary channel 9.

In an alternative and not illustrated embodiment, it is possible for the activator element 5 and the related supports 5b, while possibly being part of a single body, to be made in a distinct piece from the secondary channel.

The operation of the invention is as follows.

During inspiration, the vacuum generated inside the ampoule 2 draws air from the environment, opening a valve 10 usually present on an inlet of the secondary channel 9.

Simultaneously, the primary flow of air coming from the nozzle 6 impacts on the activator element 5, generating a turbulence that is able to create such a vacuum as to aspirate the medical liquid from the tank 8 and cause it to rise along the channels obtained on the coating body 7.

The liquid is subsequently expelled from the channels and mixed with the primary flow, generating the aerosol in correspondence with the plane of generation of the nebulisation.

The presence of the walls 9a that extend below the outlet 6a of the nozzle 6, forces the spray to follow the path indicated with the arrow B in FIG. 1 and forces the larger particles (and hence the particles with greater inertia) to resettle in the tank 8. The larger, and hence heavier, particles cannot flow by the walls 9a following the flow of air and therefore are not conveyed to the mouthpiece 3.

The secondary flow of air (whose motion is the one indicated by the arrow C in FIG. 1) also contributes to select the particles, thrusting the aerosol towards the bottom of the ampoule 2.

The air exhaled by the user is expelled by means of an additional valve 11, preferably positioned on the mouthpiece 3.

The invention achieves important advantages.

First of all, the presence of the activator element 5 physically separate from the distributor element 4 assures a primary flow of air that is substantially symmetrical relative to the axis of the primary conduit and hence assures a homogeneous dispersion of the medical liquid.

Secondly, the activator element 5, being separate from the distributor element 4, does not interfere with the fluid dynamics of the spray in correspondence with the plane of generation of the aerosol.

An additional advantage of the present invention is represented by the possibility of obtaining, on the distributor element, any number of channels. Since the activator element 5 is separate from the distributor element 4, there are no supports that may interfere with the egress of medical liquid from the channels themselves and/or with the formation of the aerosol.

Another advantage is given by the presence of the walls 9a of the secondary conduit, which allow to select the dimensions of the particles present in the spray, improving the therapeutic effect of the medical product dispensed by the ampoule 2.

1. Nebuliser ampoule (2), in particular for aerosol therapy, of the type comprising:
at least a mouthpiece (3) for dispensing a nebulised medical product;
at least an element (4) for distributing the medical product;
at least an element (5) for activating the nebulisation, physically separate from the element (4) for distributing the medical product, the distributor element (4) comprising:
at least a nozzle (6) for injecting a flow of air, called primary flow, inside the ampoule (2), said flow being necessary for generating the nebulisation;
at least a coating body (7) inserted on the nozzle (6) and provided with at least a channel for conveying the medical Product from a tank (8) of the ampoule (2) to a nebulisation area;
a supplementary, or secondary, channel (9), for introducing a flow of air, called secondary flow, into the ampoule (2) to increase and refine the nebulisation of the medical product characterised in that the coating body (7) has portions (7a) defining extensions of lateral walls (9a) of the secondary channel (9).

2. Nebuliser ampoule as claimed in claim 1,
wherein the portions (7a) consist of a ring connected to the coating body (7) by means of supporting elements (7b) and the ring is positioned in correspondence with lower ends of the lateral walls (9a),
the lateral walls (9a) together with the ring constituting means for selecting particles of the nebulisation having predetermined dimensions.

3. Nebuliser ampoule as claimed in claim 1, characterised in that the activator element (5) has a portion (5a) having substantially circular section with a flat end and is superposed to the nozzle (6) at a pre-set distance from an outlet (6a) thereof.

4. (canceled)

5. (canceled)

6. Nebuliser ampoule as claimed in claim 1, characterised in that the secondary channel (9) is coaxial to the distributor element (4).

7. Nebuliser ampoule as claimed in claim 1, characterised in that the activator element (5) is made of a single piece with the secondary channel (9).

8. Nebuliser ampoule as claimed in claim 1, characterised in that the secondary channel (9) is provided with lateral walls (9a) which extend below an outlet (6a) of the distributor element (4) or in any case below a plane of generation of the nebulisation.

9. (canceled)

10. (canceled)

11. Nebuliser apparatus, in particular for aerosol therapy, characterised in that it comprises a nebuliser ampoule (2) as claimed in any of the previous claims.
